(19) United States
(12) Patent Application Publication
(10) Pub. No.: US 2017/0215763 A1
(43) Pub. Date: Aug. 3, 2017

(54) INGESTIBLE BOLUS FOR ANIMALS

(71) Applicant: Neil Charles Helfrich, Sherwood Park (CA)

(72) Inventor: Neil Charles Helfrich, Sherwood Park (CA)

(73) Assignee: Wandering Shepherd LTD, Sherwood Park (CA)

(21) Appl. No.: 15/009,941

(22) Filed: Jan. 29, 2016

(51) Int. Cl. A61B 5/07; A01K 29/00; A61B 5/01; A61B 90/98; A61B 5/06; A01K 11/00; A61B 5/11

(52) U.S. Cl. CPC A61B 5/073; A01K 11/007; A01K 11/008; A01K 29/005; A61B 5/1112; A61B 90/98; A61B 5/062; A61B 5/01; A61B 2562/0247; A61B 2562/0219; A61B 2562/0271; A61B 2562/0204; A61B 2503/40

(57) ABSTRACT

An ingestible bolus for automatically monitoring location and physiological parameters including core temperature of ruminant animals. The location parameters are determined using magnetic field information and/or GPS data sensed by the sensors in the bolus and communicated along with a unique ID number and time stamp to a centralized location. A data center at the centralized location comprises a computing environment for analyzing the data received from the bolus and transfer the analyzed results including animal temperature, location to a user interface display. The temperature information is analyzed at the data center to determine the health status of the animal.

### BACKGROUND

1. Field of the Invention

The present invention generally relates to a system and apparatus for monitoring physiological parameters in animals. More particularly, the present invention relates to an ingestible bolus for monitoring geographical location and physiological parameters including core temperature of animals.

2. Description of Related Art

Monitoring physiological parameters of animals especially livestock is of paramount importance to ranchers, feedlot operators and dairymen. For example, dairy farmers have to monitor even subtle differences in the appearance, behavior and performance of their cows in order to respond earlier and more effectively to the animal's health problems that require immediate attention. In addition, it is imperative to know the geographical location of the animals. Generally, ruminant animal physiological parameters such as core temperature and other parameters such as unique Identification (Unique ID) number are monitored using Radio Frequency Identification (RFID) or barcode in the form of ear tag or ingestible bolus. The ingested bolus will be received in the reticulum (second pre-stomach) of the ruminant.

Monitoring the rumen can permit better management of nutrition and detection of calving. In order to measure the parameters, animal harboring the bolus have to be brought to the RFID reader or barcode reader, which involves additional labor. Handheld RFID readers or barcode readers have been developed to scan the parameters by carrying the readers to the animal site, however these devices consumes more time and also require the scanned parameters to be downloaded to a computing device for further analysis.

One of the existing problems of traditional monitoring systems include measuring the temperature of individual animals in a herd, especially when the herd is allowed to roam over a wide area. In order to monitor the temperature of an individual animal in a herd, it is necessary to correlate temperature measurements with specific animals. Several attempts have been made to provide systems for remotely monitoring the temperature of animals.

Another problem associated with the existing animal monitoring systems include lack of providing instant alert to a user regarding rise in the temperature of an animal. Instead, the user has to scan the animal using the reader to identify the modification in temperature, which is time consuming and may prove to be too late to treat the animal. Existing bolus systems include ingestible capsule comprising a transponder to send/receive animal related data to/from the reader. The reader is generally placed external to the animal and is configured to excite the transponder in order to obtain animal parameters, which is a cumbersome process. In addition, the distance within which the reader can excite the transponder is limited.

In case, if an animal is detected with abnormal temperature value, it is important to find out the exact location of the animal. Pinpointing the exact location of the animal using an automated system would save the farmer's time, which he now spends in restraining the cows, taking temperatures and recording the data. The location identification system would also save the cow from the aggravation of being separated from the herd. Especially for fresh cows, measuring physiological parameters without disturbing the animal would be a big advantage.

Therefore, there is a need for a system and device for instantly identifying the location and monitoring physiological parameters including core temperature of animals. The device allows early detection of undesirable change in physiological parameters of the animal thus facilitate timely diagnosis and treatment of diseases or sickness.

### SUMMARY OF THE INVENTION

The present invention provides a system and apparatus for automatically monitoring the core temperature, location information based on magnetic field parameters and Global Positioning System (GPS) coordinates and unique ID number of ruminant animals.

The present invention comprises an ingestible bolus, which includes specialized sensing components to monitor animals. The bolus includes a unique ID number, a magnetic field sensor for sensing an estimated magnetic field at any given point on earth at a given time and at least one data-transmitting device to send the collected data to a data center. The bolus senses, receives and transmits the unique ID number, magnetic field parameters at any given point and physiological parameters including but not limited to core temperature of the animal to a data center or a centralized location for further analysis. The bolus further comprises a Radio Frequency transponder and a Radio Frequency antenna for sending data including Unique ID, location and physiological parameters to the data center.

According to one embodiment of the present invention, the location of the animal is identified based on sensing the magnetic field parameters. The magnetic field data received from the magnetic field sensor inside the bolus is compared with a world magnetic model data at the data center. The world magnetic model includes unique magnetic field values at any point on earth's surface. By comparing the received magnetic field data with the predetermined world magnetic model data, using a reverse lookup algorithm the exact location of the animal is identified.

According to one embodiment of the present invention, in addition to magnetic field sensor, the animal's location is identified by global positioning system (GPS) and/or differential GPS. The ingestible bolus comprises one or more GPS sensors capable of transmitting GPS co-ordinates to a Global Positioning System (GPS) receiver. The GPS receiver may be used to detect both animal location and animal movement characteristics. The GPS sensor will permit determination of the location of the animal without requiring energy-consuming transmissions. Algorithms are implemented to detect the location of the animal or herd of animals. Thus, the ingestible bolus of the present invention uses the advantage of both the GPS system and magnetic field location system to obtain an even more accurate location of the animal.

The data center at a centralized location comprises a computing environment with at least one processing unit that is equipped with a control unit and an Arithmetic Logic Unit (ALU), a memory unit, a storage unit, a plurality of networking devices and a plurality Input output (I/O) devices. A software having an algorithm for manipulating the plurality of information received from the plurality of sensors inside the bolus is stored inside the storage unit and made available to the memory unit during execution of the software. The processing unit is responsible for processing the instructions of the algorithm by receiving commands from the control unit. In addition, any logical and arithmetic operations involved in the execution of the instructions are computed with the help of the ALU. The storage unit stores the software for processing the information received from the plurality of sensors in the bolus and the memory unit stores the data during run-time, while performing operations with the data received from the plurality of sensors inside the bolus.

Temperature changes of one degree Fahrenheit or even less can signal a change in the physiological condition of a ruminant animal. Further, monitoring the core temperature of ruminant animals would allow a concerned person to determine the sickness or disease. Early detection of the physiological parameters would allow the concerned person to take necessary action to prevent the spread of disease and to treat the sick or diseased animal in a timely manner, or also to monitor breeding conditions.

The objects and advantages of the embodiments herein will become readily apparent from the following detailed description taken in conjunction with the accompanying drawings.

### BRIEF DESCRIPTION OF SEVERAL VIEWS OF THE DRAWINGS

FIG. 1A illustrates an assembled view of a housing of the ingestible bolus. FIG. 1B illustrates an exploded view of the ingestible bolus housing.

FIG. 2 illustrates different modules of the bolus according to a first embodiment.

FIG. 3 illustrates different modules of the bolus according to a second embodiment.

FIG. 4 illustrates an overview of a system comprising a plurality of bolus in communication with a data center.

FIG. 5 illustrates an overview of a system comprising plurality of bolus in communication with a data center through a master device.

FIG. 6 illustrates sequential transmission of information within plurality of bolus to the data center.

FIG. 7 illustrates the components of a computing environment present in the data center.

### REFERENCE NUMERALS

100 . . . Bolus
100a, 100b, 100c . . . Bolus of first, second and third animals respectively
102 . . . Radio Frequency (RF) antenna
104 . . . RF transmitter
106 . . . RF receiver
108 . . . Power module
110 . . . Magnetic sensor module
112 . . . Temperature sensor module
114 . . . Unique ID sensor module
116 . . . GPS module
120 . . . Bolus Housing
130 . . . Closure cap of housing
200 . . . Data center
202 . . . User interface Display
220 . . . Server/Base station
300 . . . Computing Environment
302 . . . Control unit
304 . . . Arithmetic and Logical Unit (ALU)
306 . . . Processing Unit (PU)
308 . . . Networking Devices
310 . . . Input/output devices
312 . . . Memory unit
314 . . . Storage unit

### DETAILED DESCRIPTION OF DIFFERENT EMBODIMENTS

In the following detailed description, a reference is made to the accompanying drawings that form a part hereof, and in which the specific embodiments that may be practiced is shown by way of illustration. These embodiments are described in sufficient detail to enable those skilled in the art to practice the embodiments and it is to be understood that the logical, mechanical and other changes may be made without departing from the scope of the embodiments. The following detailed description is therefore not to be taken in a limiting sense.

Generally, the present invention provides a system and apparatus for determining the location and monitoring physiological parameters such as core temperature of individual animals in a herd or group of ruminant animals. The system is utilized to monitor physiological conditions of the animals spread over large distances within a location like farm, dairy facility, or a similar facility.

The present invention provides a system and apparatus for monitoring health status of individual animals or a group of animals. This monitoring is achieved by an ingestible bolus given to a ruminant animal. The ingestible bolus would settle in the stomach of the animal and senses physiological parameters including core temperature along with geolocation information of the ruminant animal. The ingestible bolus can transmit unique codes such as unique ID number along with the sensed physiological parameters such as core temperature of the ruminant animal. The bolus circuit is programmed before inserting into animal reticulum or rumen. It allows the user to program the bolus circuit with identification codes for monitoring. Moreover, the unique identification codes can be used for identification and selective manipulation of each bolus based on user requirement such as turning the bolus on and off for power requirement and modification of transmission period of a specific bolus.

Each bolus contains an active RFID microchip, which contains the unique, unalterable, fraud-proof identification code specific to each individual animal referred as unique ID (Identification) code. This unique ID code is allocated as a permanent identification number during the bolus manufacturing process. Here the communication is wireless, the bolus will communicate using RF transponder to a base station, data center and hand held user interface using wireless communication. This wireless communication is two-way since the bolus will communicate with one or more different bolus present inside other ruminant animals, and with the base station and data center. The RF transponder is configured to transmit data to and receive data from the data center or base station or a remote device. In some embodiments, this could be of one-way communication also.

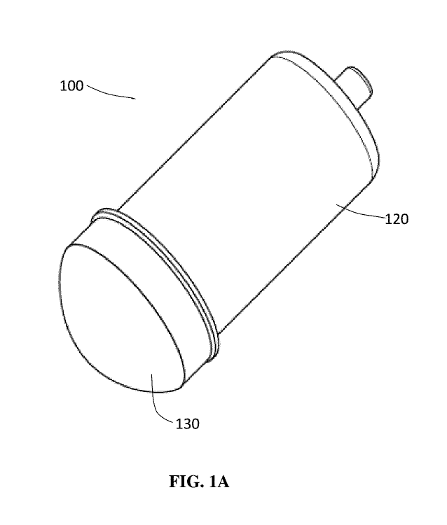

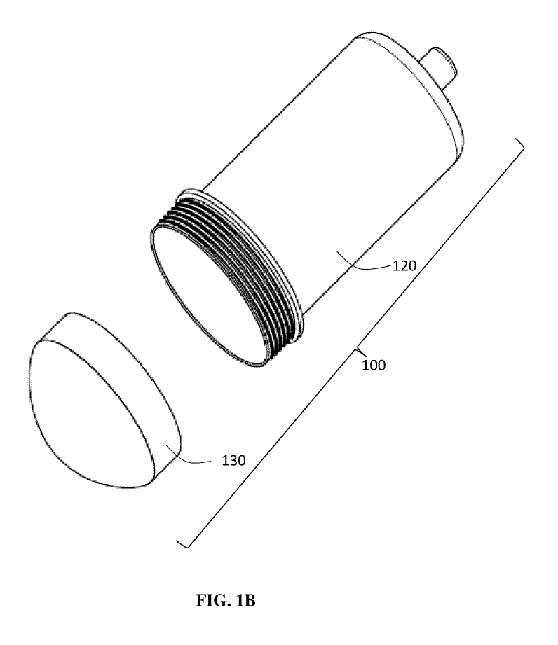

The ingestible bolus is made up of particular size and density that ensure the stability of the bolus while remaining in the animal's rumen and reticulum. Exterior surface of the bolus is made up of a suitable material to provide longer lifetime and withstand harsh environment inside the rumen of the animal without harming it. Suitable materials for manufacturing the bolus can be selected from ceramic, biochemically or medically suitable material or harmless plastic, which is inexpensive and easy for molding. FIG. 1A and FIG. 1B shows assembled and exploded views respectively, of the ingestible bolus 100 comprising a housing 120 and a closure cap 130. The bolus 100 may comprise the form of a capsule, which is configured to carry the sensing elements within it and is put inside the animal's rumen painlessly. In an embodiment, the bolus 100 comprises a dead weight to maintain the bolus 100 in a desired position within the ruminant of the animal. The bolus's unique design allows the bolus to always stand upright giving the RF antenna the best possible position at all times for the sending and receiving the RF signals.

The ingestible bolus 100 is prepared using a chemically resistant material and disposed into the animal's ruminant by administering through esophagus. The bolus 100 is made of a material that is capable to withstand the harsh environment inside the animal's rumen or stomach and thereby enabling the bolus to remain within the animal's rectum for the entire lifetime of the animal. In addition, the bolus has the ability to provide an early warning system for signs of deterioration in animal's health.

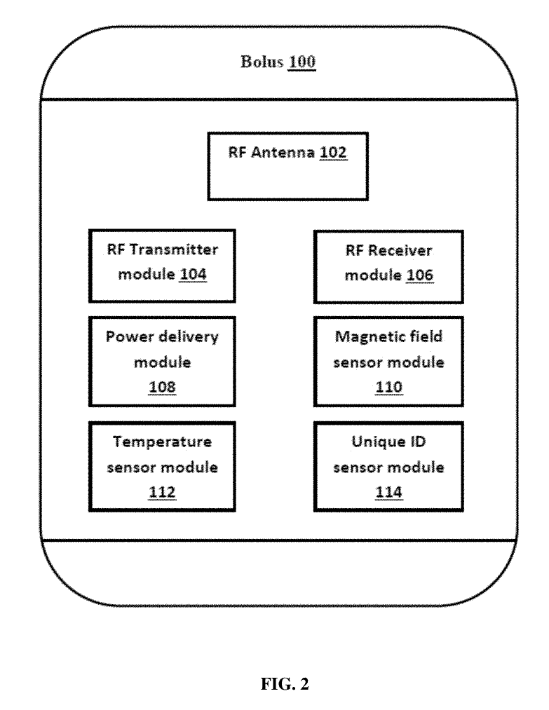

Referring to FIG. 2, according to a first embodiment, the bolus 100 comprises a wireless communicative device, which includes at least one physiological sensor such as a temperature sensor module 112 and a unique identification (ID) sensor module 114. The unique ID sensor module 114 is pre-programmed to have a unique identification code to each animal. The temperature sensor module 112 senses the core temperature of the animal continuously or at definite intervals. In order to obtain the real time position of the animal a magnetic field sensor module 110 is incorporated within the bolus 100. The magnetic field sensor module 110 provides a unique magnetic field signature corresponding to a specific location on earth's surface. The bolus 100 further comprises a RF transmitter module 104 and a RF receiver module 106, coupled with a RF antenna 102. The RF transmitter module 104 and RF receiver module 106 together is referred as RF transponder. The RF transmitter module 104 sends a plurality of information collected from the plurality of sensor modules including magnetic field sensor module 110, temperature sensor module 112 and unique ID sensor module 114, via the radio frequency (RF) antenna 102, over a wireless communications network to a centralized location. The RF receiver module 106 receives via RF antenna 102, one or more information from a data center at the centralized location.

In an embodiment, the information from the magnetic field sensor module 110 is send to the centralized location by the RF transmitter module 104 through the RF antenna 102 where it is compared with World Magnetic Model (WMM) values of the earth's field. The WMM values on earth's surface is predetermined and by comparing the measured values by the magnetic field sensor module 110 with the WMM values one can identify the latitude/longitude of the present location of the animal.

The magnetic field sensor module 110 measures the components of the magnetic field at a current location of the animal. The Earth's magnetic field measured by normal magnetic field sensor devices shows values altered by several magnetic fields on earth's surface generated by various sources. These fields interact with each other and the magnetic sensor measures the net resultant magnetic field value. In the preferred embodiment of the present invention, the measured values are free from magnetic variations by filtering the local magnetic interference due to other electronic/electrical devices by the magnetic field sensor module 110. Hence, the unique magnetic-field signature present at the animal's current location on earth can be obtained.

The bolus 100 further comprises a power delivery module 108 to supply power to the modules within the bolus 100. For example, the power module 108 can be a rechargeable battery including a button cell. The power delivery module 108 could be both active and passive. If the power delivery module 108 is active then it utilizes the internal power source such as battery present inside the bolus 100 to provide power. A battery could be an energy storage device, such as a lithium ion battery, lead acid battery, nickel cadmium battery, or the like. The power delivery module 108 could be generator such as piezoelectric generator, which generates power by the movement of the bolus 100 inside the animal. The power delivery module 108 could be also be passive, which requires stronger signals from the RF transponder comprising the RF transmitter module 104 and RF receiver module 106.

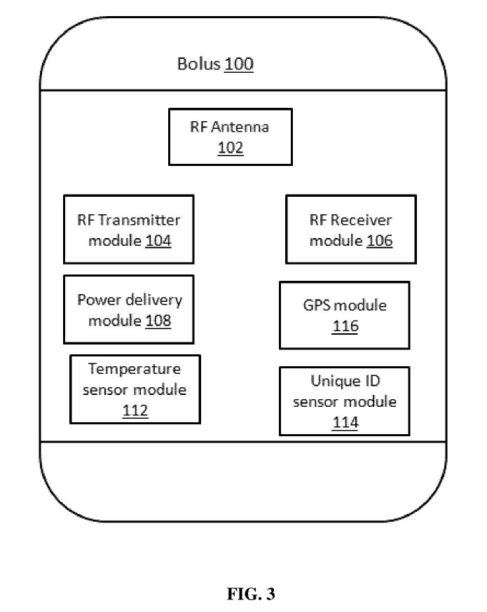

Referring to FIG. 3, according to a second embodiment, the bolus 100 comprises the Radio Frequency (RF) antenna 102 to send and receive the RF signals. Further, the bolus 100 comprises the RF transmitter module 104 and RF receiver module 106. In an embodiment, the RF transmitter module 104 sends the parameters to the centralized location or nearby bolus. In an embodiment, the RF receiver module 106 receives the RF signals from the centralized location or nearby bolus. The bolus 100 further comprises a power module 108 to supply power to the modules within the bolus 100. The bolus 100 also comprises a Global Positioning System (GPS) module 116, which tracks location coordinates of the animal. The GPS module 116 comprises one or more GPS sensors capable of transmitting GPS co-ordinates to a Global Positioning System (GPS) receiver. The GPS receiver may be used to detect both animal location and animal movement characteristics. The bolus 100 further comprises a temperature sensor module 112 and unique ID sensor module 114. In an embodiment, the temperature sensor module 112 can be a highly accurate monolithic sensor to sense the core temperature of the animal. The unique ID sensor module 114 identifies the unique identification number assigned to the individual animal.

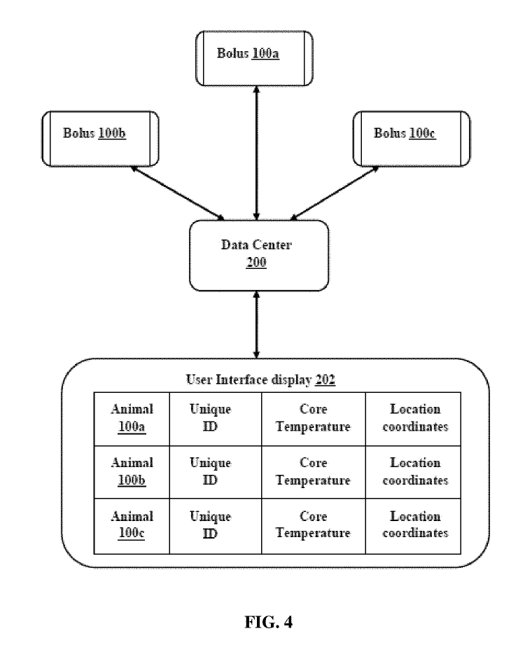

Referring to FIG. 4, which shows a system comprising a plurality of bolus in communication with a data center. Plurality of bolus 100a, 100b and 100c present in a group of animals sends the plurality of information including magnetic field data, unique ID and temperature data from the plurality of sensor modules present in each bolus. The plurality of information is transmitted using the RF transmitter module through the RF antenna. A data center 200 receives the information in signals and processes the signals to get required information regarding the animal's health information and animal's location at a particular instant. The data center 200 is configured to receive information from the plurality of bolus 100a, 100b, 100c, from different animals and process the information simultaneously based on a predefined algorithm to obtain the required details. The data center comprises to a user interface 202, which displays the unique ID, core temperature and location coordinates of each of the animals 100a, 100b and 100c.

In one embodiment, the data center 200 comprises an android operating system application to calculate the real time location of the animals from the magnetic field data received from the magnetic field sensor module 110. Android contains built-in support for the WMM using a Geomagnetic Field class. The Geomagnetic Field class utilizes the WMM internally to provide an estimated magnetic field at any given point on Earth at a given time. The class accepts the location along with altitude and time and provides the expected magnetic-field at that position, at that particular altitude and instant of time. The WMM provide a straightforward equation to generate the magnetic field for any given location. To generate a position from the magnetic readings received at the data center 200, one needs to perform an iterative reverse-search. The reverse search process starts with a default location for the first iteration and then calculating the magnetic field expected at that point using the WMM. The calculated magnetic-field parameters are mathematically deduced one and may have slight changes from the actual measured value. Since the actual measured magnetic readings received at the data center 200 from the magnetic field sensor module 110 may be affected by several magnetic fields generated by various sources, the difference between the calculated magnetic-field and the readings from the magnetic field sensor module 110 is taken. Based on the difference, a new location is decided and repeats above two steps. The process is iterated until the difference falls within acceptable limits.

A software program is designed and developed to manipulate the data received from the sensors and the WMM data. The location of the animal is estimated with the help of the software program, which is the best estimate of the present location of the animal according to the magnetic-field readings. A look-up table with pre-calculated field values for several locations across the globe is prepared, which would speed-up the search by helping in choosing a nearby location for the first iteration.

In one embodiment, the entire process of determining the location can be done on the Android device without an external connectivity using Geomagnetic Field class available in the Android operating system. The WMM provides an accurate estimate of the field of the earth's surface and is periodically updated. The magnetic field sensor module 110 can measure the components of this field and then comparing it with the WMM values of the earth's field at the data center 200 can identify the latitude/longitude of the present location. The user interface display 202 at the data center 200 shows the information from the software. The displayed information includes animals' unique ID and their health status and current location.

The direction and strength of the magnetic field can be measured at the surface of the earth and are plotted to obtain the world magnetic model. The total magnetic field is divided into several components including declination, inclination, horizontal intensity, vertical intensity and total intensity. Declination indicates the difference, in degrees, between the headings of true north and magnetic north. Inclination is the angle, in degrees, of the magnetic field above or below horizontal. Horizontal Intensity defines the horizontal component of the total field intensity. Vertical Intensity defines the vertical component of the total field intensity. Total Intensity is the strength of the magnetic field, not divided into its component parts. The Geomagnetic Field class is built upon the world magnetic model and the magnetic field sensor module 110 provides the three components of a magnetic field for a given geographical co-ordinate at a specific time. A “reverse-lookup” is implemented in the software program made available at the data center 200 to obtain the necessary location coordinates after reading a set of magnetic-field components from the magnetic field sensor module 110.

The individual magnetic field components obtained from the magnetic field sensor module 110 readings helps to pinpoint the exact location of the animal. The magnetic field sensor module 110 data from the earth's surface having magnetic field components' value, the software would change its coordinate system from the device coordinate system to the world coordinate system to compare with the world magnetic model to determine the exact location. Considering the individual magnetic field components measured by the magnetic field sensor module 110 be Bᵣ, B_{θ}, and B_{φ}, the total magnetic field value at the current location is calculated using the equation,

B_{(r,θ,φ)}=\sqrt{Bᵣ²+B_{θ}²+φ²B}

By comparing the total value of the magnetic field at a location from the world magnetic model and the received data at the data center 200 from the magnetic field sensor module 110 the software performs a reverse look up to determine the exact location of the animal.

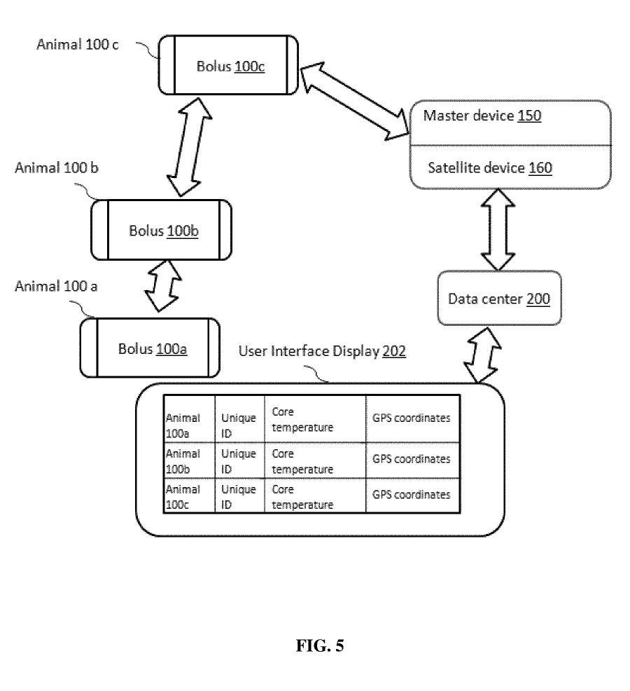

Referring to FIG. 5, showing a system comprising a plurality of the bolus 100a, 100b and 100c transferring the parameters to the data center 200 via a master device 150 which may further comprise a satellite device 160. In an example, bolus 100a sends the parameters to the adjacent bolus 100b, which in turn transfers the parameters to adjacent bolus 100c and then to the master device 150. In an embodiment, individual boluses 100a, 100b, 100c communicate among themselves in slave-slave communication functionality. From the master device 150, the parameters are transferred to the data center 200 for further analysis via the satellite device 160. The data center 200 comprises a database to store the parameters for further analysis. For example, the analysis helps to find out the exact location of the animal from the GPS coordinates; compare the core temperature of the animal with the previously retrieved core temperatures.

In an embodiment, individual boluses 100a, 100b, 100c are addressed by the master device 150 using their unique ID number. For example, the master device 150 can be a network router. In an embodiment, the master device 150 and the individual boluses 100a, 100b, 100c communicates in master-slave communication functionality and forms a tree structure. Further, the data center 200 after analyzing the parameters transfers the results to the user interface (UI) display 202. For example, the user interface display 202 can be a projector, a computer display, or the like.

In one embodiment, the data center 200 and the user interface display 202 can be remotely placed and are communicated through wireless means. In another embodiment, the data center 200 and the user interface display 202 can be located in the same place. The communication between the boluses (100a, 100b and 100c), data center 200 and user interface display 202 can be either upstream or downstream. In an embodiment, data center 200 may be communicatively coupled to a communications network including, but not limited to: a local area network (LAN), the Internet, a cellular telephone network, a telephone network, such as a Public Switched Telephone Network (PSTN), or the like.

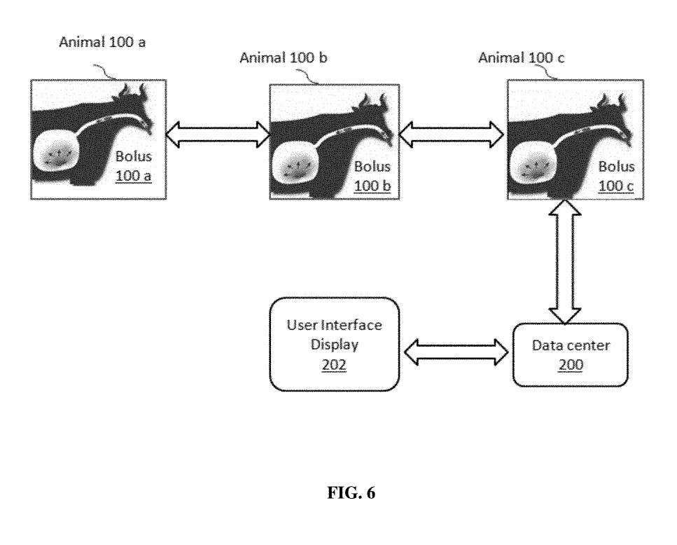

Referring to FIG. 6, showing a system comprising a plurality of bolus 100a, 100b, and 100c with respect to the animal 100a, animal 100b and animal 100c, in direct communication with a data center 200. As depicted in FIG. 6, the bolus 100a senses the parameters in the animal 100a and transfers the parameters to the nearby bolus 100b of animal 100b. Further the bolus 100b upon receiving the parameters from the bolus 100a transfers the parameters to the bolus 100c of animal 100c. The received parameters are transferred from the bolus 100c to the data center 200 for further analysis. The data center 200 comprises a database to store the parameters for analysis. For example, the analysis helps to find the exact location of the animal from the GPS coordinates or magnetic field location coordinates. Once the parameters are received at the data center 200 from the magnetic field sensor module 110 or GPS module 116, the software performs a reverse look up to determine the exact location of the animal. Similarly, the sensed core temperature is compared with the previously retrieved core temperatures. The user interface display 202 at the data center 200 shows the result of the analysis. The displayed result includes animals' unique ID and their health status and current location. In this embodiment, the bolus 100 transfers the parameters to another bolus within a range (for example up to 1.5 km) till the parameters reach a master device 150 disposed either on an animal or to a fixed object.

According to the invention, one or more sensors may be used to detect internal physiological characteristics of an animal including, but not limited to: body temperature, heart rate, blood pressure and other physiological data alike. By checking the received information on temperature of each animal, the farmers can take necessary steps to improve both the animal's health and wellbeing. Any number of sensors may be used to detect temperature and location of the animal. For example, to detect animal temperature, a temperature sensor 110 may be employed. For detection the location either magnetic sensor module 112 or GPS module 116 can be employed. It would be understood by one skilled in the sensor arts that any number of sensors, could be included within bolus 100 under the teachings presented herein. As such, this disclosure should not be construed as limited to any particular sensors.

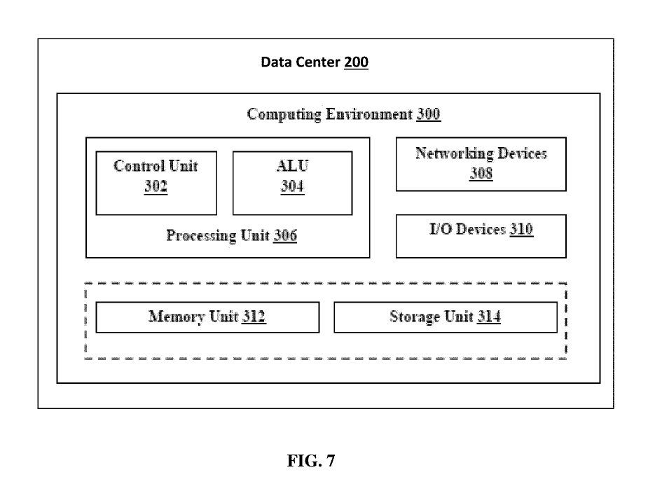

Referring to FIG. 7, showing a computing environment 300 present in the data center 200. The computing environment 300 comprises at least one processing unit 306 that is equipped with a control unit 304 and an Arithmetic Logic Unit (ALU) 302, a memory unit 312, a storage unit 314, a plurality of networking devices 308 and a plurality of Input output (I/O) devices 310. The computing environment 300 can be composed of multiple homogeneous and/or heterogeneous cores, multiple CPUs of different kinds, special media and other accelerators. Software having an algorithm for manipulating the plurality of information received from the plurality of sensors inside the bolus is stored inside the storage unit 314 and made available to the memory unit 312 during execution of the software. The processing unit 306 is responsible for processing the instructions of the algorithm. The processing unit 306 receives commands from the control unit in order to perform its processing.

Further, the plurality of processing units 306 may be located on a single chip or over multiple chips. In addition, any logical and arithmetic operations involved in the execution of the instructions are computed with the help of the ALU 304. The storage unit 314 stores the software for processing the information received from the plurality of sensors in the bolus and the memory unit 312 stores the data during run-time i.e. while performing operations with the data received from the plurality of sensors inside the bolus.

The algorithm comprising of instructions and codes required for the implementation are stored in either the memory unit 312 or the storage 314 or both. At the time of execution, the instructions may be fetched from the corresponding memory 312 and/or storage 314, and executed by the processing unit 306. Various networking devices 308 or external I/O devices 310 may be utilized for interconnecting with a variety of external devices through wireless/wired network. The computing environment 300 supports the interconnecting with the variety of external devices through the networking unit and the I/O device unit.

In above described embodiment, a programmable unique ID number may be stored on memory unit 312. In this embodiment, the unique ID number may be used to associate a bolus 100 with a particular animal. The unique ID number value may be transmitted with some or all of the messages originating from a particular bolus 100, allowing the receiver of such messages to associate the received data with a particular animal.

In one embodiment, the bolus memory may comprise read-only storage present inside memory unit 312. Read-only storage may be a Programmable Read-Only Memory (PROM), Erasable Programmable Read-Only Memory (EPROM), Electrically Erasable Programmable Read-Only Memory (EEPROM), or the like. In this embodiment, unique ID number may be stored within the memory unit 312. The unique ID number value may be transmitted with some of all of the messages transmitted from the bolus 100. In this embodiment, the unique ID number may provide a tamper-proof identifier to uniquely identify a particular bolus 100.

The foregoing description of the specific embodiments will so fully reveal the general nature of the embodiments herein that others can readily modify and/or adapt by applying current knowledge, for various applications departing from the generic concept. Therefore, such adaptations and modifications should are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit.

What is claimed is:

1. An ingestible bolus for monitoring a health status and geolocation of a ruminant animal, the bolus comprising:
a location sensor for providing a geographic location information of the ruminant animal;
a physiological sensor for monitoring at least one physiological parameter of the ruminant animal;
a unique ID for identifying each of the ruminant animal;
a Radio Frequency (RF) transponder module coupled with a RF antenna for receiving and transmitting the unique ID, geographic location information and physiological parameter to at least one of: a data center; another ingestible bolus and a remote device.

2. The ingestible bolus of claim 1, wherein said location sensor comprises a magnetic field sensor for providing a unique magnetic field signature corresponding to a geographic location of the ruminant animal.

3. The ingestible bolus of claim 1, wherein said location sensor comprises a Global Positioning System (GPS) sensor for determining GPS coordinates corresponding to a geographic location of the ruminant animal.

4. The ingestible bolus of claim 1, wherein said physiological sensor comprises a temperature sensor for measuring a core temperature of the ruminant animal.

5. The ingestible bolus of claim 1, wherein physiological sensor is selected from a group consisting of comprises a pressure sensor, vibration sensor, acceleration sensor and heat sensor

6. The ingestible bolus of claim 1, wherein the data center is configured to receive a plurality of information from the bolus and process the information based on a predefined algorithm to determine the unique ID, geographic location and physiological parameter of the ruminant animal.

7. The ingestible bolus of claim 1, wherein the remote device comprises at least one of a smartphone, mobile phone, PDA and a computer.

8. The ingestible bolus of claim 2, wherein the unique magnetic field signature is compared with the predefined WMM (World Magnetic Model) values stored in an operating system at the data center to identify the latitude/longitude of the present location of the ruminant animal.

9. A system for monitoring a health status of a ruminant animal, the system comprising:
at least one ingestible bolus configured to be disposed within a stomach of the ruminant animal, wherein the ingestible bolus comprises:
a magnetic field sensor module for providing a unique magnetic field signature corresponding to a geographic location of the ruminant animal;
a temperature sensor module for monitoring a core temperature of the ruminant animal;
a unique ID sensor module for providing a unique ID of the ruminant animal;
a Radio Frequency (RF) transponder module coupled with a RF antenna for receiving and transmitting the plurality of information comprising the unique ID, magnetic field signature and core temperature; and
a data center at a centralized location comprising a computing environment for processing the plurality of information received from the ingestible bolus based on predefined algorithm and outputting results comprising geographic location, health status associated with the unique ID of the ruminant animal on a user interface.

10. The system of claim 9, further comprises a remote device connected to the data center through a communication network.

11. The system of claim 10, wherein the remote device comprises at least one of a smartphone, mobile phone, PDA and a computer.

12. The system of claim 9, further comprises a master device or a satellite device for receiving the plurality of information from a plurality of ingestible bolus and transferring the plurality of information to the data center.

13. The system of claim 12, wherein the plurality of information is sequentially transferred between the plurality of ingestible bolus to reach the master device or a satellite device or to reach the data center.

14. The system of claim 9, further comprises a Global Positioning System (GPS) sensor module for determining GPS coordinates corresponding to a geographic location of the ruminant animal.
